(19) United States
(12) Patent Application Publication
(10) Pub. No.: US 2015/0377872 A1
(43) Pub. Date: Dec. 31, 2015

(54) MEDICAL DIAGNOSTIC TEST SYSTEMS, AND A MATRIX THEREFOR

(71) Applicant: WHATMAN GMBH, Dassel (DE)

(72) Inventors: Klaus HOCHLEITNER, Dassel (DE); Suzana KIEL, Dassel (DE); Alexander SCHENK, Dassel (DE); Wei SUN, Dassel (DE)

(21) Appl. No.: 14/766,397

(22) PCT Filed: Feb. 3, 2014
(86) PCT No.: PCT/EP2014/052051

§ 371 Date: Aug. 6, 2015

(30) Foreign Application Priority Data
Feb. 8, 2013 (GB) 1302292.6

(51) Int. Cl. G01N 33/543

(52) U.S. Cl. CPC G01N 33/5436

(57) ABSTRACT

The invention discloses a reduced thickness membrane matrix for carrying liquids consisting of a cellulosic material with a thickness of between 10 and 50 μm and a pore size between 0.1 μm and 20 μm for use with diagnostic tests such as lateral flow tests, thereby reducing the amount of test reagents required.

### FIELD OF THE INVENTION

Embodiments of the present invention relate to medical diagnostic test systems and a liquid carrier matrix for use with such a system.

### BACKGROUND

Many countries have limited financial resources for healthcare and few highly trained healthcare workers. Low cost diagnostic tests which are easy to interpret are therefore very attractive to such country's healthcare organisations. Medical diagnostics, in particular, is often expensive, and the necessary equipment often requires training and skill to operate, as well as often needing a clean environment to operate. The development of cheap biomedical devices that can be easily operated would be greatly appreciated not only within resource-limited nations, but also in emergency situations where time is of the essence and specialized equipment is not immediately available. Paper-based biomedical devices have been developed to address this need. However, they have a number of limitations. One limitation is that the chemicals commonly used to prepare these devices are toxic. Another limitation is that conventional filter paper cannot be used to immobilize proteins in a way that does not damage the proteins, and is therefore unsuitable for many standard biomedical assays such as the western blot. (Analytical Chemistry, 82 (1), 329-335 DOI).

A large number of rapid diagnostic test systems are currently based on the combination of mostly cellulosic membranes with papers and/or glass fiber pads. The most dominant test system available on the marketplace is the so-called “lateral flow test” (see e.g Posthuma-Trumpie et al. Anal. Bioanal Chem (2009), 393, pp 569 582, and references cited therein).

The system allows for the rapid diagnosis of very diverse physiological conditions for example: fertility; infectious diseases; drugs of abuse; marker molecules for cardiac issues and the like from bodily fluids.

The diagnostic test is in most cases be performed at a point of care, but it some cases even a layperson may run the diagnostic test at home (e.g. pregnancy and fertility tests).

The membrane materials currently available on the marketplace range in thickness between about 100 and about 150 μm. This membrane layer needs to be filled completely with capture reagents although the signal that is usually being generated by colored particles linked to detector reagents is visible only for a part of the membrane thickness (Rapid Lateral Flow Test Strips, Millipore Corporation (2008)), thus resulting in an unnecessary waste of precious capture reagents, and as a consequence, manufacturing costs higher than necessary.

Therefore, there is a need to provide a liquid carrying membrane matrix that consistently reduces the reagent without compromising the analytical signal, combined with a superior consistency and reproducibility of the diagnostic test result.

In rapid diagnostic tests using conventional membranes with visual indications, including colorimetry, chemiluminiscence, fluorescence and the like, the signal that is visible to the test user is generated within the first few micrometers of the membrane surface. Any signal that is generated deep within the matrix does not contribute to the visible signal. Nevertheless, the whole membrane volume underneath the surface needs to be filled with reagents, and the whole membrane volume needs to be filled with sample liquid when the test is being used. This is an unnecessary waste of reagents which can be very expensive depending on the test system under consideration. Also the amount of sample liquid consumed is high, which is problematic where the sample volume is low.

### SUMMARY

In an embodiment, a membrane matrix material is provided that has an effective thickness of far less than 100 μm, with a consistency in terms of thickness and capillary flow, for example a membrane having a thickness variance of 5% or less and a capillary flow with a variance of 10% or less.

The membrane can be manufactured in a dry cast process by forming a liquid layer of a casting mix consisting of nitrocellulose, organic solvents, water and surfactants onto a moving support, and then forming a thin membrane on the support by evaporating the solvents in the formed layer, under controlled conditions using a counter-current air or gas flow. The porosity of the membrane can be controlled by adjusting the water content of the casting mix to required percentages varying between 5 and 15% (v/v).

In an embodiment, for ease of handling, the membrane is left on the support on which it has been formed. Other options are the use of a fleece material in the casting mix to stabilize the forming membrane, or to laminate the membrane onto self-adhesive supports. The thickness of the casting mix layer on top of the support and the consistency of this thickness needs to be controlled very carefully. Prior to the evaporation, for the new thin membrane materials, the liquid layer of casting mix is adjusted to values below 100 μm whereas for conventional membranes, the thickness of the layer is usually a couple of hundred micrometers, more specifically around 800 μm which requires less accurate control.

This thin casting mix layer also demands for a very exact control of heat, humidity, speed of the moving support and of the countercurrent gas stream.

In an embodiment, a diagnostic test is provided that uses the thin membrane mentioned above. Thereby said test has the same sensitivity/specificity/runtime properties as a diagnostic test using conventional membrane materials with a thickness of 100-150 μm or more, but capture reagent volume needs of 30 50% or less compared to conventional membrane reagent volume needs.

### BRIEF DESCRIPTION OF THE DRAWINGS

FIG. 1 shows a scanning electron microscope (SEM) image of the surface of the thinner membrane, at 500× magnification. The image shows a nitrocellulose membrane consisting of a meshwork of nitrocellulose fibers without surface impurities; and

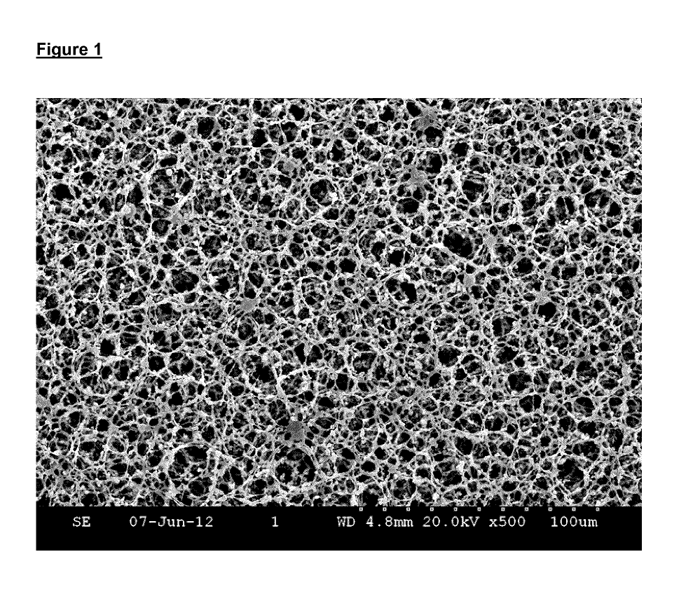

FIG. 2 shows a picture and analysis of a pregnancy test at the limit of detection with the thinner membrane after use in a test strip, as obtained by reflectance spectroscopy. All pads were removed prior to analysis by reflectance spectroscopy.

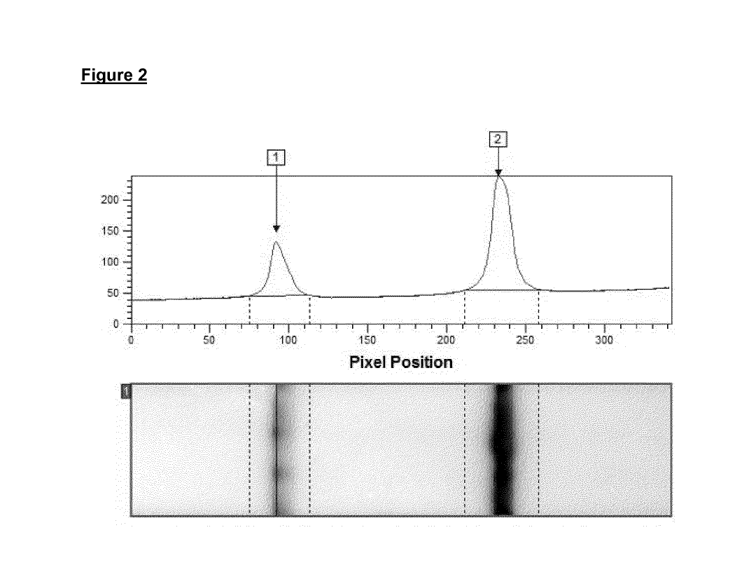

### DETAILED DESCRIPTION

The following description of the exemplary embodiments refers to the accompanying drawings. The following detailed description does not limit the invention. Instead, the scope of the invention is defined by the appended claims.

Diagnostic Test Example:

A generally conventional pregnancy diagnostic test was prepared using conventional paper sample pads and wicks, and glass fibre conjugate pads for all test strips investigated, while the thinner membranes mentioned above were used in this setup. The detector reagent used was a mouse anti-beta-hCG antibody conjugated to 40 nm colloidal gold. The capture antibody is a mouse anti-alpha-hCG antibody. hCG hormone was diluted into the sample liquid in order to obtain the required final concentration. The membrane test strip width was 5 mm. Test line results were obtained using a reflectance reader.

Thin Membrane Example:

Thin membranes with a capillary flow of about 125 seconds/4 cm (test liquid: water) were obtained by using a casting mix comprising:

| | |
|---|---|
| Ethanol | 39.9% |
| Methylacetate | 38.4% |
| Nitrocellulose | 11.7% |
| Water | 9.9% |
| Surfactants | 0.1% |

These membranes were manufactured by moving a support surface under the casting mix and depositing a layer of the mix onto the support. In this case, the casting mix was poured onto a 100 μm PET foil support at a casting speed of 30 cm/min and a maximum casting mix thickness of 90 μm and the membrane was formed by drying the mix in a conventional manner, to form a membrane having a final mean thickness of 43 μm plus the thickness of the foil.

The pore size was approximately 8 μm. Membranes with a smaller or larger pore size (0.1 μm to 20 μm) was obtainable by decreasing or increasing the water content of the casting mix. Additional membranes were cast resulting in a dried membrane thickness of 25 μm, (without foil thickness). For all membranes produced, the thickness variability was less than 5%-coefficient of variance (CV), and capillary flow time variability was less than 10% (CV). It was determined that the minimum thickness for a membrane using the manufacturing process mentioned above is around 10 μm.

The membranes were used in the pregnancy test described above, and the amount of test line antibody required to detect hCG at a concentration of 25 mIU/ml was determined for the new membranes as well as conventional PET-backed NC membranes with similar capillary flow time properties as shown in table 1.

TABLE 1
Capture line reagent requirements on different membranes

| | Mean Thickness of NC | Amount of capture antibody |
|---|---|---|
| | Layer without backing foil | required to detect 25 mIU |
| Test Membrane | (μm) | hCG/ml sample (ng) |
| New membrane | 25 | 100 |
| New membrane | 43 | 175 |
| GE FF 120 HP | 100 | 300 |
| Competitor 1 | 120 | 400 |
| Competitor 2 | 120 | 400 |

The examples shown above illustrate that thinner membranes can be manufactured using generally conventional so-called dry casting technologies, and that they need less (up to 80% less) reagent as compared to conventional membranes when used in lateral flow diagnostic test. It follows that the same is true for flow-through diagnostic test systems, line assays and Western Blots were liquids flow through the volume of the membrane laterally (in a planar manner) or vertically-(surface to surface) in a generally capillary or forced manner. Such membranes enable the manufacturing of rapid tests at lower costs, and allow for miniaturisation of test systems. It has been shown that there is no reduction in test performance.

One example of a membrane has been described above, and one example of a diagnostic test employing the membrane has been described, it will be appreciated that other membranes could be manufactured, and those membranes could be employed in other tests, all within the scope of the claims.

For example, a PET foil membrane support is described. Other supports include other foils such as metal foils or other plastics foils such as PVC or polystyrene; integrated fleeces or webs, such as non-woven polyester webs; fibrous materials such a glass fibre materials, or papers, for example high quality chromatography papers.

A range of pore sizes between 0.1 and 20 μm has been described above, specific sub-ranges within the 0.1 and 20 μm range will be useful, for example 2 to 20 μm for use with lateral flow diagnostic tests where capillary fluid flow is important and where the 8 μm pore size example given above is about right. In other examples the sub range is 2 to 6 μm for use in a lateral flow diagnostic test where the cost of reagents is high and so as little fluid, and reagent, as possible is used for an assay. All other sub ranges or specific values within the 0.1 to 20 μm are possible.

1. A membrane matrix for carrying liquids consisting of cellulosic material(s) with a thickness of between about 10 μm and about 50 μm and a pore size between about 0.1 μm and about 20 μm allowing the flow of liquids.

2. The membrane matrix of claim 1 comprising Nitrocellulose.

3. The membrane matrix of claim 1, wherein the matrix has a thickness of between 10 μm and 30 μm.

4. The membrane matrix of claim 1, wherein the cellulosic material is unsupported following casting.

5. The membrane matrix of claim 1, wherein the cellulosic material is supported by an integrated fleece during casting.

6. The membrane matrix of claim 1, wherein the cellulosic material is supported by an integrated web during casting.

7. The membrane matrix of claim 1, wherein the membrane is formed on a solid support during casting on which it remains for further use.

8. The membrane matrix of claim 7, wherein the solid support includes a plastic foil.

9. The membrane matrix of claim 7, wherein the solid support includes a paper.

10. The membrane matrix of claim 7, wherein the solid support includes a glass fibre material.

11. The membrane matrix of claim 7 wherein the support includes a metal foil.

12. The membrane matrix of claim 7, wherein the solid support comprises any combination of a plastic foil, a paper, a glass fibre material, and/or a metal foil.

13. The membrane matrix of claim 4, wherein the membrane is fixed onto a support material by means of an adhesive, flowing said casting.

14. A medical diagnostic test comprising:
the membrane matrix of claim 1 for carrying liquids;
reagents absorbed into said membrane matrix to capture target molecule(s) and/or to use as reporter reagents whose presence is to be detected in a liquid sample flowing through said membrane matrix; and
liquid absorbent elements adjacent the membrane matrix.

15. A medical diagnostic test as claimed in claim 14, wherein the reagent is present in a
volume which is 30% less than the volume used in an equivalent diagnostic test employing a membrane having a thickness of 100 μm or more.

16. A medical diagnostic test as claimed in claim 14, wherein the reagent is present in a
volume which is 50% less than the volume used in an equivalent diagnostic test employing a membrane having a thickness of 100 μm or more.

17. A medical diagnostic test as claimed in claim 14 wherein the reagent is present in a
volume which is 80% less than the volume used in an equivalent diagnostic test employing a membrane having a thickness of 100 μm or more.

18. (canceled)
